(19) United States
(12) Patent Application Publication
(10) Pub. No.: US 2026/0363590 A1
(43) Pub. Date: Nov. 19, 2026

(54) EMOTIONAL STATE DETECTION

(71) Applicant: British Telecommunications Public Limited Company, London, Greater London (GB)

(72) Inventors: Ian OAKLEY, London (GB); Max Smith-Creasey, London (GB)

(21) Appl. No.: 19/155,774

(22) PCT Filed: Jan. 8, 2024
(86) PCT No.: PCT/EP2024/050297

§ 371 Date: Aug. 12, 2025

(30) Foreign Application Priority Data
Feb. 15, 2023 (EP) 23156783.5
Feb. 15, 2023 (GB) 2302143.9

(51) Int. Cl. A61B 5/16; A61B 5/00; A61B 5/11

(52) U.S. Cl. CPC A61B 5/165; A61B 5/0077; A61B 5/1123; A61B 5/4803; A61B 5/6898; A61B 5/7246; A61B 5/7267; A61B 5/7278

(57) ABSTRACT

A computer implemented method for detecting an emotional state of a user by using non-personally identifying user actions. The method comprising detecting at least one non-personally identifying user action and determining the emotional state of the user that corresponds to the detected non-personally identifying user action. This is done using a first model that associates non-personally identifying user actions with corresponding emotional states and a pre-existing second model that determines a user's emotional state based on received biometric data, the second model being trained by machine learning.

### TECHNICAL FIELD

The present invention relates to mood or emotional state detection. In particular, the present invention relates to a computer implemented method for detecting an emotional state of a user by using non-personally identifying user actions.

### BACKGROUND

The use of biometrics to infer an individual's emotional state is well-known. There is significant research to show that by detecting biometrics such as an individual's facial expressions, tone of voice, or heartrate, their emotional state can be inferred. For example, see the paper “Recognition of Emotion Intensities Using Machine Learning Algorithms: A Comparative Study” by Dhwani Mehta, Mohammad Faridul Haque Siddiqui, and Ahmad Y. Javaid, PMCID: PMC6514572.

It is also well-known that an individual's emotional state can be utilised in mood based recommendation systems. For example mood based music recommendation systems may use biometric data to infer an individual's emotional state. With a combination of artificial intelligence technologies, biometric data and generalised music therapy approaches, a recommendation system can be generated to aid the individual with music selection for different life situations and maintain their mental and physical conditions.

As biometrics are Personally Identifying Data (PID), these recommendation systems can compromise the individual's privacy. This can give rise to issues relating to the handling of personal information such as under General Data Protection Regulation (GDPR).

Accordingly, improvements to these methods that do not compromise the individual's privacy are desirable.

### SUMMARY OF INVENTION

The invention is defined in the independent claims. Optional features are set out in the dependent claims.

Embodiments relate to a method for detecting an emotional state of a user by using non-personally identifying user actions. The method comprises: detecting at least one non-personally identifying user action; and determining the emotional state of the user that corresponds to the detected non-personally identifying user action. The emotional state is determined by inputting information indicative of the detected user action into a first model that associates non-personally identifying user actions with corresponding emotional states. The first model is generated by determining correlations between, on the one hand, emotional states that have been previously determined using biometric data and, on the other hand, previously detected non-personally identifying user actions.

Non-personally identifying user actions are considered to be actions for which the detected data required to detect such an action does not indicate or identify a specific individual. Examples of these actions include gestures (such as raising a hand) or interactions with a user device (such as ordering food on an app, or asking a smart speaker to play a specific song).

Decoupling the detected emotions from the biometric data can be performed using non-personally identifying user actions to generate a depersonalised model, which is beneficial for preserving an individual's privacy. Compared to detection of emotional states using biometrics, detection of emotional states with non-personally identifying user actions, sometimes referred to as environment-derived emotion detection methods, requires less precise data gathering, and thus can be less invasive (e.g., a closed-circuit television (CCTV) camera vs an iris scanner).

Advantageously, methods that detect emotional states with non-personally identifying user actions involve observing actions rather than specific individual characteristics and therefore, minimise the amount of identifying data collected, and thus enable models to be constructed of action-emotion relations without needing any Personally Identifying Data (PID), for example as per the General Data Protection Regulation (GDPR) or equivalent.

The methods described establish a link between actions and emotions. Therefore, if the individual's emotional state can be inferred (from biometric data or non-personally identifying user actions) their subsequent actions can be predicted and anticipated.

Optionally, the emotional states that have been previously determined using biometric data are determined by using a pre-existing second model that determines a user's emotional state based on received biometric data, the second model being trained by machine learning.

Optionally, the biometric data is data which contains information that can be used to personally identify the user such as facial images or vocal properties.

Optionally, the non-personally identifying user action is an action, the identifying data for which cannot be used to clearly identify a user, such as a gesture, or selection of media content.

Optionally, the non-personally identifying user action is detected using a microphone, camera or user device such as a mobile phone or tablet device.

Optionally, the method further comprises controlling one or more smart devices to carry out a function based on the determined emotional state of the user and one or more rules. The rules are predetermined and link emotional states to functions carried out by one or more smart devices. For example, a rule may link a particular emotional state to a function carried out by controlling a media device to play a piece of media determined based upon the determined emotional state of the user.

Optionally, the method further comprises determining a subsequent emotional state of the user after carrying out the function and updating the one or more predetermined rules if a desired change in emotional state is not determined. In some embodiments, this method minimises the period of time that biometric data is collected over and eventually, does not require biometric data.

Advantageously, updating the predetermined rules improves the model's recommendation capabilities. This is because the recommended function is intended to alter the emotional state of the user. By redetermining the user's emotional state after the function has been carried out, the model can verify whether or not the desired change has been achieved. If it has not, the model will be updated so it does not recommend that function again in the circumstances.

Optionally, the method further comprises detecting the emotional states of one or more additional users in a common environment by, for each additional user: detecting at least one non-personally identifying user action; determining the emotional state of the user that corresponds to the detected non-personally identifying user action by inputting information indicative of the detected user action into the first model.

Methods that detect emotional states with non-personally identifying user actions evaluate the environment as a whole, including all individuals in the environment rather than specific individuals. Therefore, group utility can be used as a policy parameter without requiring aggregation of individual emotions.

Optionally, the method further comprises determining the presence of each user via detection means configured to detect the presence of a plurality of users. For example the presence of each person in the vicinity of the detecting device, such as within a room or building, may be detected.

Optionally, the method further comprises controlling one or more smart devices to carry out a function based on the determined emotional states of the plurality of users. In particular, the method may control one or more smart devices to carry out a function based on the determined emotional states of the plurality of users and one or more rules. In a similar manner to that described above, the rules are predetermined and link emotional states to functions carried out by one or more smart devices. For example, a rule may link a set of emotional states to a function carried out by controlling a media device to play a piece of media determined based upon the determined emotional states of the users.

Advantageously, the system can seek to maximise mood utility by detecting the various emotional states of the users in the environment and recommending a function to optimise for mood.

As a further aspect, according to embodiments, a method of training a model using machine learning is provided, the trained model being capable of determining the emotional state of a user based on their non-personally identifying actions. Training data is applied to the model. The training data comprises (i) emotional states that have been previously determined using biometric data and (ii) previously detected non-personally identifying user actions. The model is trained according to a machine learning algorithm to identify correlations between the non-personally identifying user actions and emotional states.

The machine learning algorithm that is used can for example be supervised, unsupervised or reinforcement based, including one or more of:
Naïve Bayes
Support Vector Machine
Linear Regression
Logistic Regression
Decision Tree
Random Forest
K-Nearest Neighbour
K-Means Clustering
Artificial Neural Networks.

As the model is trained to identify correlations between non-personally identifying user actions and emotional states occurring over a common period of time, the biometric data used to identify the emotional states is not required by the model at this stage. In some embodiments, the method further does not require the detection of biometric data at all after the model reaches a pre-set level of accuracy in identifying the correlation between a non-personally identifying user actions and an emotional state. This is beneficial as it minimises the amount of Personally Identifying Data that is used and proactively preserves people's privacy.

Optionally, the previously determined emotional states and the non-personally identifying user actions are contemporaneous.

Optionally, there is provided a computer model that has been trained using the aforementioned training method.

According to a further aspect, there is provided a computer system comprising a processor and a memory storing computer program code configured to carry out any of the methods described above.

### BRIEF DESCRIPTION OF THE DRAWINGS

The disclosure will be further described, by way of example only, with reference to the accompanying drawings, in which:

FIG. 1 is a flowchart illustrating an overview of the described method;

FIG. 2 is a flowchart illustrating a method of determining an emotional state using a non-personally identifying user action;

FIG. 3 is a flowchart illustrating a method of training a model using machine learning to determine an emotional state of a user by using non-personally identifying user actions; and

FIG. 4 is a flowchart illustrating an example of the system on which the method operates.

### DETAILED DESCRIPTION OF THE INVENTION

Embodiments and related technology helpful for understanding and implementing the embodiments will now be described with reference to the Figures. The same or similar reference numerals are used to refer to the same or similar components across different Figures.

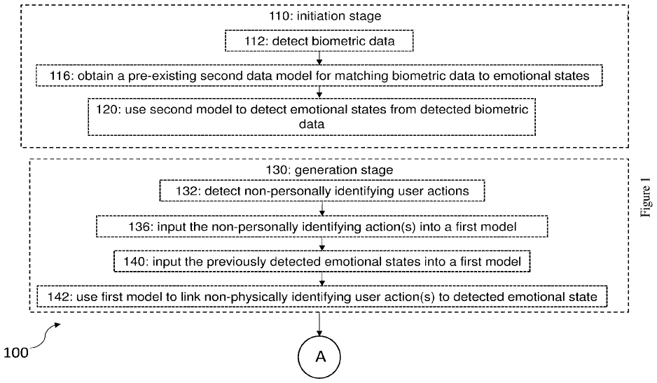

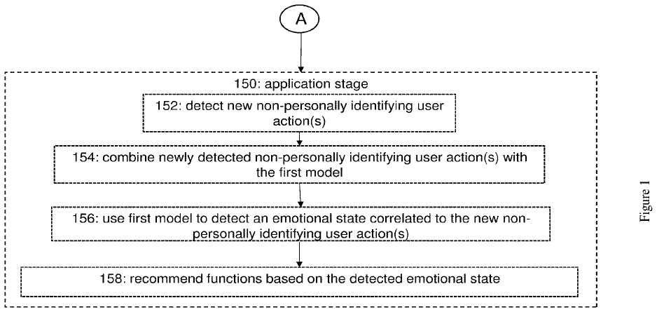

FIG. 1 shows the overall method 100 of detecting an emotional state 118 using one or more non-personally identifying user actions 134. A non-personally identifying user action 134 is sometimes termed an environmental biometric and includes a variety of actions carried out by a user such as a physical gesture or interacting with media content. For example, a non-personally identifying user action 134 could be asking a smart speaker to play a specific song or giving someone a thumbs up.

Generally an initiation stage is used to detect emotional responses by users according to established methods that use biometric data, i.e. a set of emotions are detected using physical biometrics. A generation stage then uses the detected emotional responses to train a model that recognises emotional responses from user actions, i.e. once there is a set of detected emotions to compare against, a model can be generated by comparing observed user actions to observed emotions. This generation stage may iterate with continuous addition of fresh data until a sufficiently complete model is generated. An application stage uses the trained model from the generation stage to detect emotional responses and to carry out or recommend one or more functions performed by smart devices.

The initiation stage 110 of the model begins with the step of detecting biometric data 112. Biometrics, or biometric data. 114 are known to be measurable, distinctive characteristics of a human which can be used to label and describe individuals. Individuals can therefore be identified using one, or a combination, of their biometrics 114. Biometrics 114 include physiological characteristics. The detected biometrics 114 can for example comprise one or more of:
Facial images,
Pulse measurements,
Gait measurements,
Breathing pattern measurements,
Chemical signature measurements (e.g. from breath and/or perspiration),
Voice recordings,
Handwriting scans,
Handling signature measurements (e.g. one or more of orientation, direction and/or speed and/or acceleration of translational and/or rotational motion, holding pressure, frequency of interaction and/or changes in and/or patterns of changes in one or more of these)
User interface interaction signature measurements (e.g. characteristic ways of one or more of typing, pressing buttons, interacting with a touch sensitive or gesture control device and viewing a display, for example determined through one or more of: force and pressure on a tactile interface; speed, rhythm, frequency, style and duration of interaction with a tactile or gesture based interface; and visual tracking of a display), and
Linguistic analysis measurements (e.g. from free text type and/or voice recordings).

To determine what emotional state 118 the user is in, a pre-existing second data model 122 is used to perform step 120. The second model 122 could for example be obtained from, or implemented in, the cloud. This data model 122 uses machine learning to match biometrics 114 to emotional states 118. For example, if an individual's facial images show they are crying, the model will detect the emotional state 118 of sadness. Such models are known in the art, as are the various manners of generating and training them. For example, see the paper “Recognition of Emotion Intensities Using Machine Learning Algorithms: A Comparative Study” referenced in the Background section above.

Once a learned range of emotional states 118 have been determined, the generation stage 130 begins by detecting non-personally identifying user actions 132. Information indicative of these non-personally identifying user actions 134 is inputted 136 to a first model 138. The biometric data 114 used in the initiation stage to determine corresponding emotional responses and the non-personally identifying user actions 134 occur within the same time frame or time period. The detected emotion corresponding to the biometric data, determined according to the second model, is also inputted 140 into the first model 138. The first model 138 uses a machine learning algorithm to identify a correlation 142 between the non-personally identifying user actions 134 and the detected emotional state 118 without factoring in the biometric data 114. Continuing the above example, the first model 138 may take, as an input, the detected emotional state 118 of sadness. In addition an input is provided identifying the non-personally identifying user actions 134 occurring at the time the emotional state of sadness was determined by the second model. For example a correlation may be found between the user asking their smart speakers to play a specific song and the emotion of sadness being determined. In this example, the first model 138 would identify a correlation between the requested song and the emotional state 118 of sadness, but not with the facial image from which the expression indicative of crying is determined.

Once the first model 138 has been trained to cover a sufficiently broad range of emotional states 118, the application stage 150 can begin. This stage 150 concerns using the first model 138 to detect emotional states 118 and recommend functions based on the detected emotional state 158. According to the application stage, new non-personally identifying user actions 134 are detected 152. These new non-personally identifying user actions 134 are, at step 154, input into the first model 138. As explained above, the first model 138 is used to detect an emotional state correlated to the new non-personally identifying user actions 156. Depending on the emotional state 118 detected, the first model 138 will subsequently recommend a function 158. For example, if the first model 138 has detected the emotional state 118 of sadness, it might recommend adding tissues to a list maintained by a smart shopping app. The steps of the method 100 can loop indefinitely, as the computer system 170 compares new non-personally identifying user actions 132 to the learned range of emotional states 114.

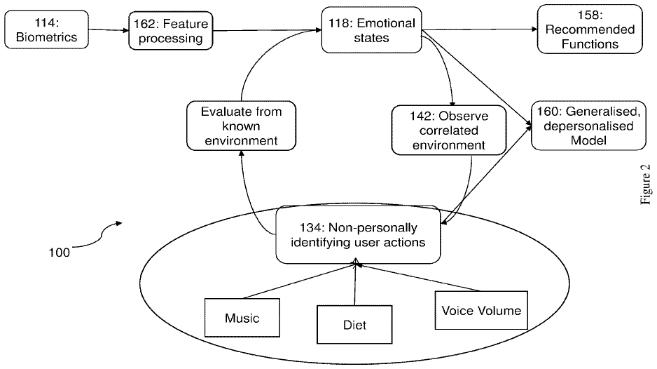

FIG. 2 illustrates, in an alternative form, the method 100 of generating a depersonalised model 160 to detect emotional states 118 using non-personally identifying user actions 134 and subsequently recommend functions 158. Starting from the left, biometrics 114 are detected and their characteristic features are processed 162 using a pre-existing data model 122 to determine emotional states 118 as explained above. Non-personally identifying user actions 134 are also detected. A variety of examples of non-personally identifying user actions 134 are provided in FIG. 2. For example, a particular choice of media content, such as music, might be detected by an individual choosing to play a particular artist, song and/or genre. For example, one or more properties of a user's diet might be detected by a camera 404 monitoring the food an individual takes out of their fridge or detected by what they order on their mobile device or tablet 172. For example, voice volume might be detected by a microphone.

FIG. 2 shows how the first model 138 is trained and used to match emotional states 118 to non-personally identifying user actions 134. The right side of the loop demonstrates the training of the first model 138 to identify the correlation between emotional states 118 and observed correlated non-user identifying actions 134. The left side of the loop illustrates using the trained model to take a non-personally identifying user action 134 and detect a correlated emotional state 118.

In the embodiment depicted in FIG. 2, the first model is a generalised depersonalised model 160 because it can detect emotional states 118 using non-personally identifying user actions 134 without the need for the biometric data 114 after the learned range of emotional states 118 has been established.

The other output of the flowchart in FIG. 2 illustrates that the system can recommend functions 158 based on the detected emotional states 118 as detailed above. Once a function has been recommended 158, a reinforcing feedback loop can be set up between the non-personally identifying user actions 134 and generalised, depersonalised model 160. New non-personally identifying actions 134 can be detected to determine an emotional state 118 resulting from the recommended function 158. This provides feedback to the model 160, as the predetermined rules can be reenforced (if the resulting emotional state 118 had the intended effect) or changed (if the resulting emotional state 118 did not have the intended effect).

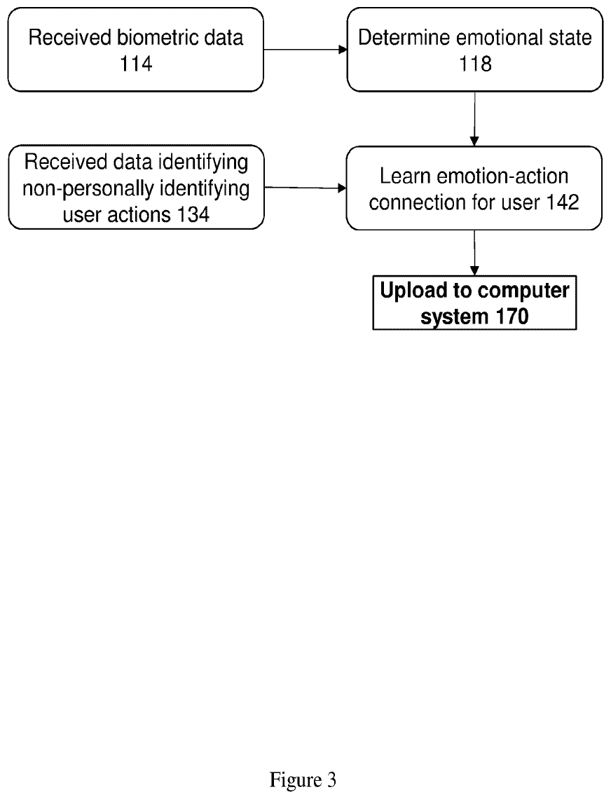

FIG. 3 illustrates a method of training the first model 138 to identify the correlation between actions 134 and emotions 118. Two sets of data are applied to the model:
1. Previously determined emotional states 118 (that have been determined based on biometric data as described above)
2. Previously detected non-personally identifying user actions 134
   The model is trained according to a machine learning algorithm. The machine learning algorithm that is used can for example be one of:
Naïve Bayes
Support Vector Machine
Linear Regression
Logistic Regression
Decision Tree
Random Forest
K-Nearest Neighbour
K-Means Clustering
Artificial Neural Networks
   Methods of training such a model are known in the art and will not be elaborated on here.
   Once the model 138 has learnt the emotion-action connection, this is uploaded to the computer system 170.

As indicated in FIG. 3, according to the learning process for generating the first model, physical biometrics are found and their corresponding emotions are also found. The user actions that accompanied the biometrics (and the corresponding determined emotional response) are found. The physical biometrics are then stripped out leaving a logical connection between emotion and user action, which is learned and uploaded. By stripping out the physical biometrics, any PID is also removed, thus ensuring that in a mature trained system user privacy is retained.

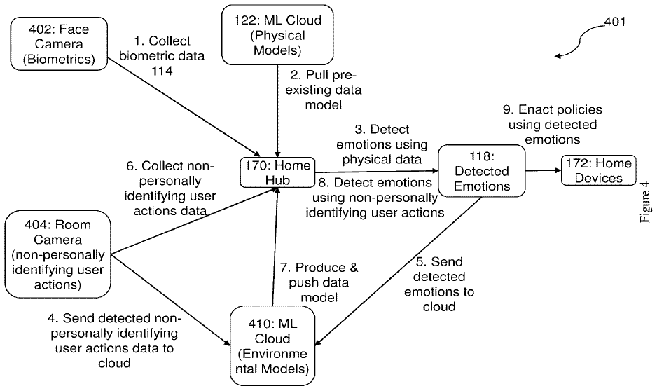

FIG. 4 illustrates an example of the system 401 on which the method of FIG. 2 operates. To eventually determine emotional states 118 using non-personally identifying user actions 134, the system first detects biometric data 114 using a sensor device. In this example, a face camera 402 is used to detect one or more facial expressions of the user, such as smiling or crying. Other devices for detecting biometric data may be used such as a microphone or pulse monitoring equipment for example.

The system also uses a sensor device or user input device to detect non-personally identifying user actions 134. In this example, a room camera 404 is used to detect one or more gestures/physical actions of the user, such as raising a hand. Other devices for detecting non-personally identifying user actions may include smart devices having user input mechanisms for causing the smart devices to carry out particular actions such as selecting media content or making online purchases.

A user computing device 170 receives the biometric data 114 and may use a pre-existing data model 122 to determine emotional states 118 from the detected biometrics 114. The user computing device may be a home hub, being a device that connects devices 172 on a home automation network and controls communications among them

In this example, the pre-existing data model 122 is a physical model pulled from data storage such as a machine learning cloud or remote server for example. The determined/detected emotional states may be sent to a machine learning system 410, such as a cloud based system, for further processing to produce the first model.

In addition, the machine learning system 410 receives the detected non-personally identifying user actions data and uses this in combination with the detected/determined emotions 118 to produce the first model as described above.

The user computing device 170 receives future user action data from the room camera 404 and uses the trained first model, obtained from the machine learning system 410, to detect emotions using the non-personally identifying user actions.

The detected emotions 118 are then used to recommend functions 158. In this example, the system instructs home devices 172 (such as smart speakers and apps) to enact policies based on the detected emotions 118.

Specific uses of the described method will now be described.

A first example relates to music selection. There has been much work done in the prior art showing that correlations can be found between a given piece of music, the intended emotional effect of the music, and the observed emotional effect on a given listener. Therefore, it is possible to use this correlation in the home environment for emotion detection purposes.

A stereotypically sad piece of music might be played on a home sound system. Using physical biometrics collected from devices such as a room camera, and a physical-to-emotional trained machine learning (ML) model using an algorithm such as an artificial neural network (i.e. a second model according to the description above, being a model that associates biometrics with emotions) it can be computationally determined what emotion the listener is feeling at that time.

Over a long enough sample range, regularly observing how the listener is feeling physically when listening to a given track, and what tracks the listener listens to when observably sad, a new model (i.e. a first model according to the description above) can be incrementally trained that demonstrates this correlation. Once this new model is sufficiently broad the physical biometrics can be dispensed with entirely and these listening habits can be used as a sole indicator of listener emotions.

With this model, other actions can be anticipated. For example, if the individual is asking their smart speaker for music the system knows they treat as sad, then the user can be identified as sad and thus, the system can make related recommendations such as recommending that tissues are added to the shopping list of the linked smart shopper app.

A second example relates to detecting the emotional state of multiple users. For the purpose of this example, it is noted that gestures are classed as a non-personally identifying user action because they are not Personally Identifying Information. In a conversation, individuals express their points, and their emotions, with the aid of their body language. These emotions can be expressed in a range of gestures such as; a raised fist, a middle finger, a hand covering the mouth. These can be used as signifiers of emotion even without knowing what the individual is saying, once a personalised link between emotion and the individual's gesture has been identified.

For example, a user computer system or device, such as a smart home hub, listening for voice commands detects two individuals in conversation: person A and person B. At the same time, person A has their arms crossed and, in response, person B has their head in their hands. From the vocal context, these indicate a disagreement, that the two individuals are annoyed, and that this is how they express their annoyance.

Therefore, a machine learning algorithm such as a KNN (K-nearest neighbour) can be used to identify the patterns in how an individual uses their hands in a disagreement. Once enough data has been collected, it is possible to predict whether an individual is annoyed purely by using their non-personally identifying actions (how they move their hands and what gestures they make), without needing to record what they say. If person A is crossing their arms, this means they are annoyed. The system can recommend functions to improve the mood upon detecting this. For example, the smart speaker can use this information to play music known to be calming for person A and person B and thus defuse the situation.

A third example relates to diet. There is a cultural concept known as “comfort food”, foodstuffs such as stews or burgers eaten when an individual is unwell or sad. Similarly, alcohol consumption is often linked to emotional state-an individual may drink with friends and be happy whereas drinking alone can be a sign of sadness or depression.

As such, these habits can also be used for detecting emotional states using non-personally identifying actions. A group of people are observed drinking wine together by a room camera. The room camera can identify the smiles on their faces and the smart home hub (as in the previous example) can note the tones of their voices, and together verify that the individuals in the group are each happy. A first model according to the description above can make the link between the group drinking wine together and the emotional state of happiness. With enough examples, the biometrics can again be dispensed with entirely, and the system can recommend that the smart speaker play a happy playlist (as defined by the user's revealed preferences) to accompany this happy environment.

Other embodiments will be apparent to those skilled in the art from consideration of the specification and practice of the embodiments disclosed herein. It is intended that the specification and examples be considered as exemplary only.

In addition, where this application has listed the steps of a method or procedure in a specific order, it could be possible, or even expedient in certain circumstances, to change the order in which some steps are performed, and it is intended that the particular steps of the method or procedure claims set forth herein not be construed as being order-specific unless such order specificity is expressly stated in the claim. That is, the operations/steps may be performed in any order, unless otherwise specified, and embodiments may include additional or fewer operations/steps than those disclosed herein. It is further contemplated that executing or performing a particular operation/step before, contemporaneously with, or after another operation is in accordance with the described embodiments.

The methods described herein may be encoded as executable instructions embodied in a computer readable medium, including, without limitation, non-transitory computer-readable storage, a storage device, and/or a memory device. Such instructions, when executed by a processor (or one or more computers, processors, and/or other devices) cause the processor (the one or more computers, processors, and/or other devices) to perform at least a portion of the methods described herein. A non-transitory computer-readable storage medium includes, but is not limited to, volatile memory, non-volatile memory, magnetic and optical storage devices such as disk drives, magnetic tape, compact discs (CDs), digital versatile discs (DVDs), or other media that are capable of storing code and/or data.

Where a processor is referred to herein, this is to be understood to refer to a single processor or multiple processors operably connected to one another. Similarly, where a memory is referred to herein, this is to be understood to refer to a single memory or multiple memories operably connected to one another.

The methods and processes can also be partially or fully embodied in hardware modules or apparatuses or firmware, so that when the hardware modules or apparatuses are activated, they perform the associated methods and processes. The methods and processes can be embodied using a combination of code, data, and hardware modules or apparatuses.

Examples of processing systems, environments, and/or configurations that may be suitable for use with the embodiments described herein include, but are not limited to, embedded computer devices, personal computers, server computers (specific or cloud (virtual) servers), hand-held or laptop devices, multiprocessor systems, microprocessor-based systems, set top boxes, programmable consumer electronics, mobile telephones, network personal computers (PCs), minicomputers, mainframe computers, distributed computing environments that include any of the above systems or devices, and the like. Hardware modules or apparatuses described in this disclosure include, but are not limited to, application-specific integrated circuits (ASICs), field-programmable gate arrays (FPGAs), dedicated or shared processors, and/or other hardware modules or apparatuses.

User devices can include, without limitation, static user devices such as PCs and mobile user devices such as smartphones, tablets, laptops and smartwatches.

Receivers and transmitters as described herein may be standalone or may be comprised in transceivers. A communication link as described herein comprises at least one transmitter capable of transmitting data to at least one receiver over one or more wired or wireless communication channels. Wired communication channels can be arranged for electrical or optical transmission. Such a communication link can optionally further comprise one or more relaying transceivers.

User input devices can include, without limitation, microphones, buttons, keypads, touchscreens, touchpads, trackballs, joysticks and mice. User output devices can include, without limitation, speakers, buzzers, display screens, projectors, indicator lights, haptic feedback devices and refreshable braille displays. User interface devices can comprise one or more user input devices, one or more user output devices, or both.

1. A computer implemented method for detecting an emotional state of a user by using non-personally identifying user actions, the method comprising:
detecting at least one non-personally identifying user action; and
determining the emotional state of the user that corresponds to the detected non-personally identifying user action by inputting information indicative of the detected user action into a first model that associates non-personally identifying user actions with corresponding emotional states;
wherein the first model is generated by determining correlations between (i) previously determined emotional states that have been determined based on biometric data and (ii) previously detected non-personally identifying user actions.

2. The method of claim 1, wherein the previously determined emotional states that have been determined based on biometric data are determined by:
using a pre-existing second model that determines a user's emotional state based on received biometric data, the second model being trained by machine learning.

3. The method of claim 1, wherein the biometric data is data which contains information that can be used to personally identify the user such as facial images or vocal properties.

4. The method of claim 1, wherein the non-personally identifying user action is an action, the identifying data for which cannot be used to clearly identify a user, such as a gesture, or selection of media content.

5. The method of claim 1, wherein the non-personally identifying user action is detected using a microphone, camera or user device such as a mobile phone or tablet device.

6. The method of claim 1, further comprising controlling one or more smart devices to carry out a function based on the determined emotional state of the user and one or more predetermined rules.

7. The method of claim 6 wherein carrying out the function comprises:
controlling a media device to play a piece of media determined based upon the determined emotional state of the user.

8. The method of claim 6, further comprising determining a subsequent emotional state of the user after carrying out the function and updating the one or more predetermined rules if a desired change in emotional state is not determined.

9. The method of claim 1 further comprising detecting the emotional states of one or more additional users in a common environment by, for each additional user:
detecting at least one non-personally identifying user action;
determining the emotional state of the user that corresponds to the detected non-personally identifying user action by inputting information indicative of the detected user action into the first model.

10. The method of claim 9 further comprising determining the presence of each user via detection means configured to detect the presence of a plurality of users.

11. The method of claim 9, further comprising controlling one or more smart devices to carry out a function based on the determined emotional states of the plurality of users.

12. A computer system comprising a processor and a memory storing computer program code for performing the steps of claim 1.

13. A method of training a model using machine learning to determine an emotional state of a user by using non-personally identifying user actions, the method comprising:
applying training data to the model, the training data comprising (i) previously determined emotional states that have been determined based on biometric data and (ii) previously detected non-personally identifying user actions;
training the model according to a machine learning algorithm to identify correlations between the non-personally identifying user actions and emotional states.

14. The method of claim 13 wherein:
the previously determined emotional states occurred within respective specified time periods and the previously detected non-personally identifying user actions occurred in one of the respective specified time periods.

15. A computer model trained according to the method of claim 13.
